(19) United States
(12) Patent Application Publication
(10) Pub. No.: US 2025/0099678 A1
(43) Pub. Date: Mar. 27, 2025

(54) WIRELESS COMMUNICATION MANAGEMENT IN INFUSION PUMP SYSTEMS

(71) Applicant: Tandem Diabetes Care, Inc., San Diego, CA (US)

(72) Inventors: Josh Juster, San Diego, CA (US); Geoffrey Kruse, San Diego, CA (US)

(21) Appl. No.: 18/891,482

(22) Filed: Sep. 20, 2024

Related U.S. Application Data
(60) Provisional application No. 63/584,305, filed on Sep. 21, 2023.

(51) Int. Cl. A61M 5/172; A61B 5/145; G16H 40/67; H04W 4/38; H04W 52/02

(52) U.S. Cl. CPC A61M 5/1723; A61B 5/14532; G16H 40/67; H04W 52/0241; H04W 4/38

(57) ABSTRACT

Disclosed herein are methods for establishing communication protocols between wireless devices in infusion pump systems. Infusion pump systems can include a number of components capable of wireless communication with one or more other components including an infusion pump, a continuous glucose monitoring (CGM) system and a smartphone or other multi-purpose consumer electronic device. Communications among these devices can be coordinated to increase efficiency and conserve battery power.

### PRIORITY CLAIM

This application claims the benefit of U.S. Provisional Application No. 63/584,305, which is hereby incorporated herein by reference in its entirety.

### TECHNICAL FIELD

The present disclosure is directed to portable infusion pumps and more particularly to wireless communications in infusion pump systems.

### BACKGROUND

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type 1, or in some cases, type 2 diabetes. Some insulin injecting pumps are configured as portable or ambulatory infusion devices can provide continuous subcutaneous insulin injection and/or infusion therapy as an alternative to multiple daily injections of insulin via a syringe or an insulin pen. Such pumps are worn by the user and may use replaceable cartridges. In some embodiments, these pumps may also deliver medicaments other than, or in addition to, insulin, such as glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Ambulatory infusion pumps have generally been controlled by a user interface provided on the pump. With the proliferation of handheld electronic devices, such as mobile phones (e.g., smartphones), there is a desire to be able to remotely utilize such devices, as well as dedicated wireless controllers designed to work with one or more infusion pumps and/or types of infusion pumps, to optimize usage of infusion pumps. These remote controllers would enable a pump to be monitored, programmed and/or operated more privately, more conveniently and more comfortably. Accordingly, one potential use of dedicated remote devices and handheld consumer electronic devices (such as smartphones, tablets and the like) is to utilize such devices as controllers for remotely programming and/or operating infusion pumps.

However, portable infusion pumps are generally powered by portable batteries and frequent wireless communications with external devices can have a significant effect on infusion pump battery life. In addition to mobile control devices such as smartphones and dedicated remote controllers it may be beneficial to enable infusion pumps to communicate with other devices, such as continuous glucose monitors, glucose meters, and other health monitoring devices, for example. This places further burden on infusion pump battery life. It would therefore be desirable to configure such communications in a manner that reduces the strain on the infusion pump battery and otherwise increase the efficiency of such systems.

### SUMMARY

Disclosed herein are methods for establishing communication protocols between wireless devices in infusion pump systems. Infusion pump systems can include a number of components capable of wireless communication with one or more other components including an infusion pump, a continuous glucose monitoring (CGM) system and a smartphone or other multi-purpose consumer electronic device. Communications among these devices can be coordinated to increase efficiency and conserve battery power.

In embodiments, a method of coordinating wireless communications in an infusion pump system including a continuous glucose monitoring (CGM) device, an infusion pump and a remote electronic device is disclosed herein. A communication session can be initiated with the CGM device in which the CGM device measures glucose levels of a user for calculation of therapy parameters for delivery of insulin to the user with the infusion pump. It can then be determined if the infusion pump is communicatively coupled with the remote electronic device. Glucose levels can be transmitted from the CGM device to the remote electronic device if the infusion pump is communicatively coupled with the remote electronic device and from the CGM device to the infusion pump if the infusion pump is not communicatively coupled with the remote electronic device.

In embodiments, a method of coordinating wireless communications in an infusion pump system including a continuous glucose monitoring (CGM) device, an infusion pump and a remote electronic device is disclosed herein. A communication session can be initiated with the CGM device in which the CGM device measures glucose levels of a user for calculation of therapy parameters for delivery of insulin to the user with the infusion pump. If it is determined that the infusion pump is communicatively coupled with the remote electronic device, no communications occur between the infusion pump and the CGM device.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 depicts an embodiment of a pump system according to the disclosure.

FIG. 2 depicts a block diagram representing an embodiment of a pump system according to the disclosure.

FIGS. 3A-3B depicts an embodiment of a pump system according to the disclosure.

FIG. 4 depicts an embodiment of a pump system according to the disclosure.

FIGS. 5A-5B depict remote control devices for a pump system according to embodiments of the disclosure.

FIG. 6 depicts a schematic representation of a pump system according to an embodiment of the disclosure.

FIG. 7 depicts a flowchart of methods steps for managing wireless communications in an infusion pump system according to an embodiment of the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

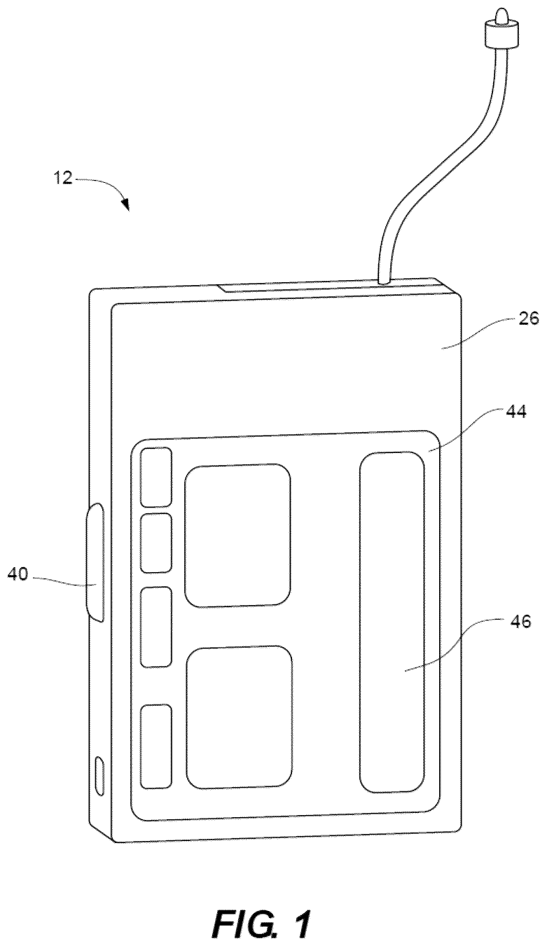

FIG. 1 depicts an exemplary medical device that can be used with embodiments of the disclosure. In this embodiment, the medical device is configured as a pump 12, such as an infusion pump, that can include a pumping or delivery mechanism and reservoir for delivering medicament to a patient and an output/display 44. The type of output/display 44 may vary as may be useful for a particular application. The output/display 44 may include an interactive and/or touch sensitive screen 46 having an input device such as, for example, a touch screen comprising a capacitive screen or a resistive screen. The pump 12 may additionally include a keyboard, microphone, or other input device known in the art for data entry, which may be separate from the display. The pump 12 may also include a capability to operatively couple to one or more blood glucose meters (BGMs) or continuous blood glucose monitors (CGMs) and/or one or more secondary devices such as a remote display, a remote control device, a laptop computer, personal computer, tablet computer, a mobile communication device such as a smartphone, a wearable electronic watch, smart ring, electronic health or fitness monitor, or personal digital assistant (PDA), a CGM display etc.

In one embodiment, the medical device can be a portable pump configured to deliver insulin to a patient. Further details regarding such pump devices can be found in U.S. Pat. No. 8,287,495, which is incorporated herein by reference in its entirety. In other embodiments, the medical device can be an infusion pump configured to deliver one or more additional or other medicaments to a patient.

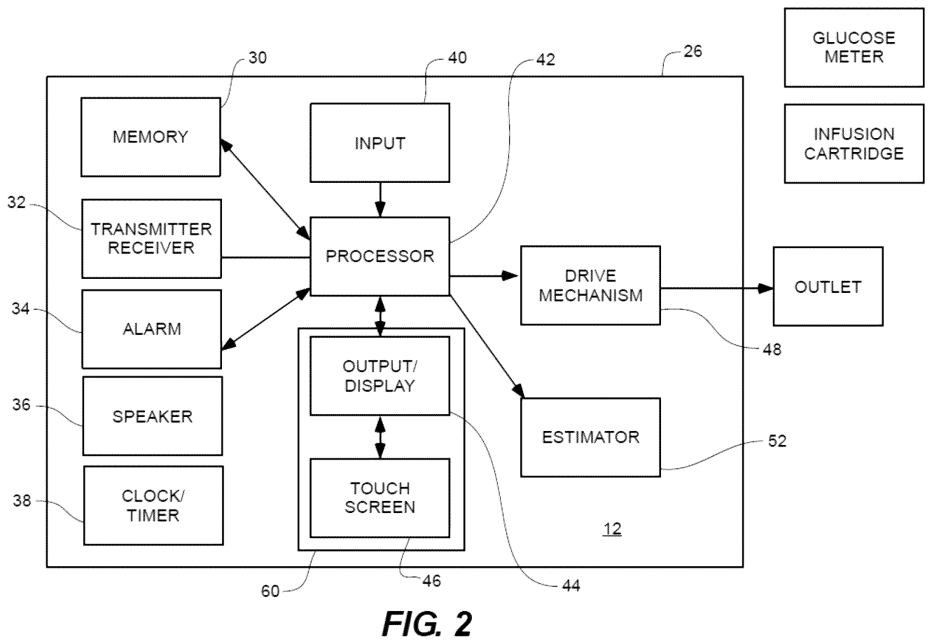

FIG. 2 illustrates a block diagram of some of the features that can be used with embodiments, including features that may be incorporated within the housing 26 of a medical device such as a pump 12. The pump 12 can include a processor 42 that controls the overall functions of the device. The infusion pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface as shown in FIG. 2 is a graphical user interface (GUI) 60 having a touch sensitive screen 46 with input capability. In some embodiments, the processor 42 may communicate with one or more other processors within the pump 12 and/or one or more processors of other devices, for example, a continuous glucose monitor (CGM), display device, smartphone, etc. through the transmitter/receiver. The processor 42 may also include programming that may allow the processor to receive signals and/or other data from one or more input devices, such as sensors that may sense pressure, temperature and/or other parameters.

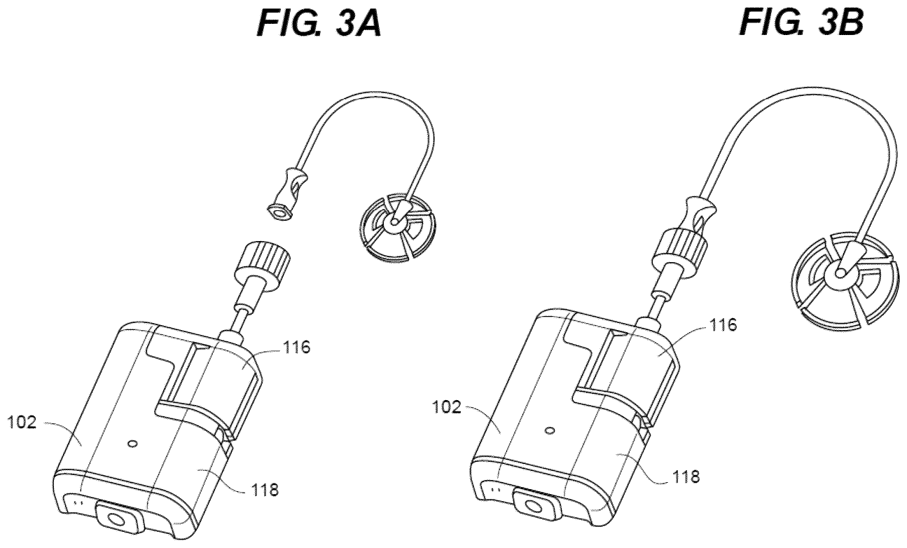

FIGS. 3A-3B depict a second infusion pump that can be used in conjunction with one or more embodiments of the ambulatory infusion pump system of the present disclosure. Pump 102 includes a pump drive unit 118 and a medicament cartridge 116. Pump 102 includes a processor that may communicate with one or more processors within the pump 102 and/or one or more processors of other devices such as a remote device (e.g., a CGM device), a remote control device, or a consumer electronic device (e.g., laptop computer, personal computer, tablet computer, smartphone, electronic watch, smart ring, electronic health or fitness monitor, or personal digital assistant). The processor 42 may also include programming to receive signals and/or other data from an input device, such as, by way of example, a pressure sensor, a temperature sensor, or the like. Pump 102 also includes a processor that controls some or all of the operations of the pump. In some embodiments, pump 102 receives commands from a separate device for control of some or all of the operations of the pump. Such separate device can include, for example, a dedicated remote control device or a consumer electronic device such as a smartphone having a processor executing an application configured to enable the device to transmit operating commands to the processor of pump 102. In some embodiments, processor can also transmit information to one or more separate devices, such as information pertaining to device parameters, alarms, reminders, pump status, etc. Such separate device can include any remote display, remote control device, or a consumer electronic device as described above. Pump 102 can also incorporate any or all of the features described with respect to pump 12 in FIG. 2. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. Further details regarding such pumps can be found in U.S. Pat. No. 10,279,106 and U.S. Patent Publication Nos. 2016/0339172 and 2017/0049957, each of which is hereby incorporated herein by reference in its entirety.

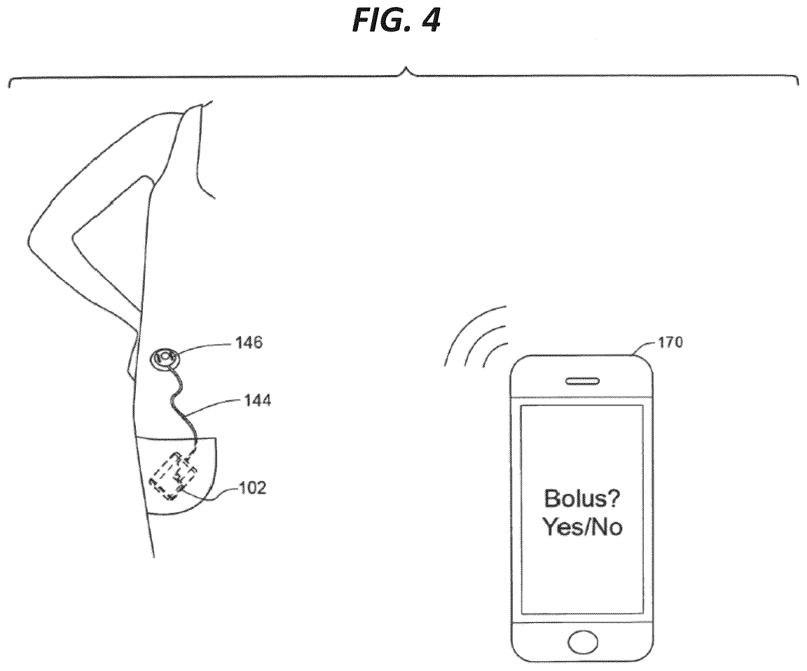

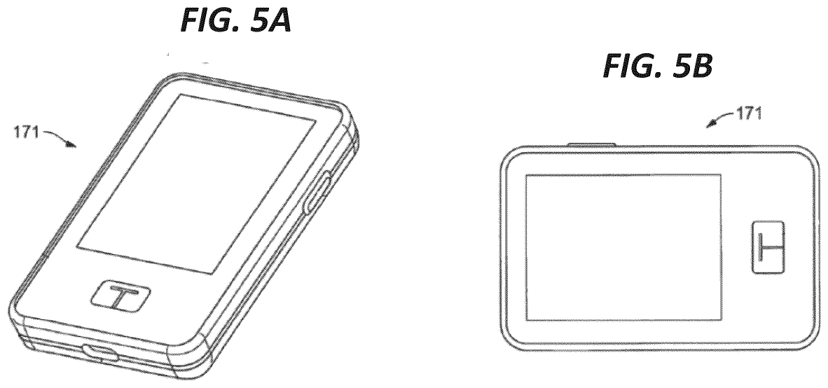

Referring to FIGS. 4-5B, one or more remote control devices 170, 171 can be used to communicate with the processor of pump 12 and/or pump 102 to control delivery of medicament and transfer data with pump via a wired or a wireless electromagnetic signal, such as via, e.g., a near field communication (NFC) radio frequency (RF) modality or other RF modalities such as Bluetooth®, Bluetooth® low energy, mobile or Wi-Fi communication protocols, for example, according to embodiments of the present disclosure. Such a remote control can include, for example, a mobile communication device 170, such as a smart phone (as depicted in FIG. 4) executing a software application for control of the pump, a dedicated remote controller 171 (as depicted in FIGS. 5A-5B), a wearable electronic watch, smart ring, electronic health or fitness monitor or personal digital assistant (PDA), etc., or a tablet, laptop or personal computer. Such communications between (and among) the one or more remote control devices 170, 171 and pump 102 may be one-way or two-way for, e.g., effective transfer of data among the devices and the pump, control of pump operations, updating software on the devices and/or pump, and allowing pump-related data to be viewed on the devices and/or pump.

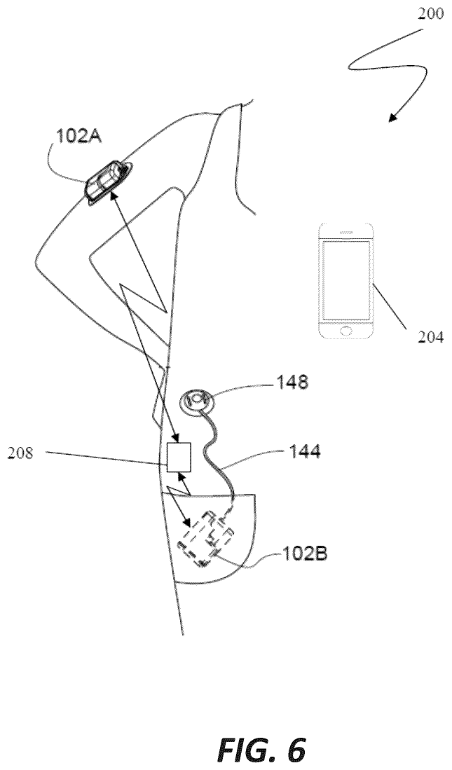

FIG. 6 depicts a schematic representation of a pump system 200 according to an embodiment. System 200 includes a user-wearable infusion pump such as pump 12 or pump 102 described above. In embodiments, a user can alternatively wear the pump 102A directly on the body or place the pump 102B in the user's pocket or other location near the body with infusion tubing 144 extending to an infusion set 148 on the user's body. The system 200 also includes a continuous glucose monitoring (CGM) sensor with a corresponding transmitter 208. The CGM sensor obtains measurements relating to glucose levels in the body and the transmitter communicates that information to the pump 102A/B. The pump 208 can then use the glucose data in making therapy determinations. The system can also include a one or more devices such as a smartphone 204 or other multi-purpose consumer electronic device capable of operating a software application to communicate with and/or control the pump and, alternatively or additionally, a dedicated remote control device designed specifically for use with pump 102A/102B. The smartphone 204 or other remote control device can in some embodiments also be capable of communication with CGM sensor/transmitter 208. In addition, the pump 102A/B and/or smartphone 204 or other remote control device can optionally communicate with additional devices such as, for example, a blood glucose meter or other analyte sensing device, an activity or other health monitor, etc.

Although depicted with the multi-purpose consumer electronic device 204 being a smartphone, in various embodiments the consumer electronic device can alternatively or additional include one or more of a wearable electronic watch, such as a smartwatch, a smart ring, electronic health or fitness monitor, personal digital assistant (PDA), or a tablet, laptop or personal computer, etc. A multi-purpose consumer electronic device can be any device sold to consumers and used for a variety of functions and which can be configured or programmed to communicate with and/or control an infusion pump as one of said functions. In some embodiments, systems as described herein may include more than one multi-purpose consumer electronic device configured for communication with the infusion pump (e.g., a smartphone and a smart watch).

One concern in pump systems 200 such as the system depicted by way of example in FIG. 6 is that the multiple communication options for the pump can lead to an excessive drain of the pump's rechargeable battery and inefficient and/or redundant communications. Commonly owned U.S. Patent Publication No. 2020/0213946, which is hereby incorporated herein by reference, addresses similar issues and includes disclosure relating to coordinating communications among a smartphone, dedicated remote controller and pump to conserve battery power of a pump. The present application provides additional novel communication methods that can aid in reducing the battery drain on a pump while still providing all the advantages of such an integrated communication system, particularly with regarding to communications related to the CGM device in the system.

As noted above, pump 102 can receive glucose information from the CGM system 208 and control commands from the smartphone 204, for example. Pump 102 can also send communications to these devices. In embodiments, the devices communicate using Bluetooth. Pump 102 is generally continually in communication with CGM system 208 to receive information pertaining to glucose levels and other data. For example, the CGM system 208 may provide data every five minutes to the pump 102. Pump 102 may be generally regularly communicating with the CGM system 208 on a periodic basis and continuously or periodically broadcasting an advertisement data packet for detection by the phone 204 and/or other devices when not already connected to these devices.

The use of the pump to continually scan for and/or be in communication with the CGM is power intensive for the pump. As such, embodiments disclosed herein are designed to reduce the burden on the pump (and therefore the pump battery) for such communications. For example, when the pump is connected to a phone or other remote device, the pump can cease automatically scanning (e.g., broadcasting an advertisement data packet) for and/or communicating directly with the CGM. In some embodiments, when the pump is connected to the phone or other device, the pump does not attempt to establish a wireless connection with the CGM because the pump knows that the phone or other device is the default device for communication with the CGM. The phone can instead establish communications with the CGM. Only if the pump is not in communication with such a device do the CGM and pump directly communicate. In this manner, drain on pump battery for such communications and corresponding therapy calculations is avoided as the pump need only receive a corresponding therapy control command from the phone or other device based on the CGM communications. Although primarily described with a smartphone 204 being the remote communication device, it should be understood that any other device capable of wireless communications with a pump and a CGM described herein can operate as the device that communicates with the CGM, such as, for example, a dedicated remote control device, wearable electronic (smart) watch, smart ring, etc.

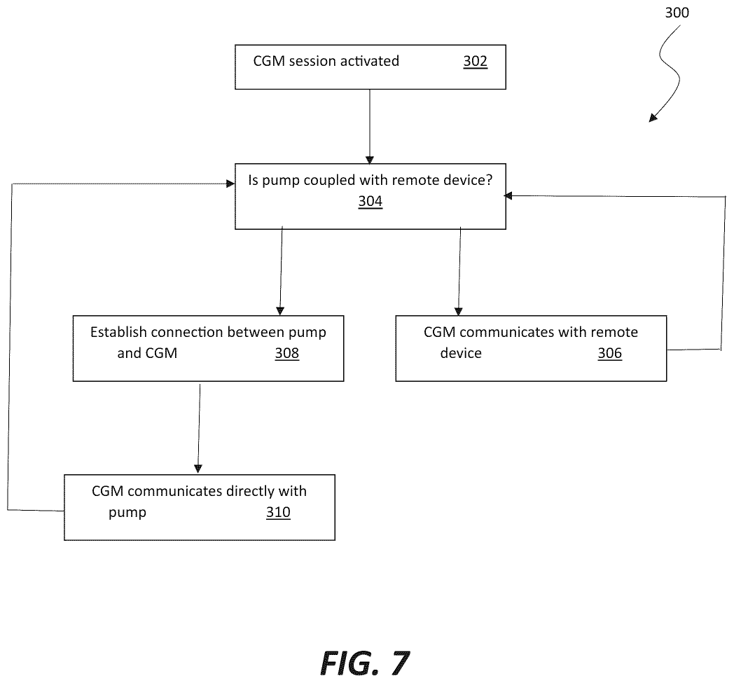

Referring now to FIG. 7, a flowchart of method steps for a method 300 of coordinating wireless communications in an infusion pump system is depicted. At step 302, a CGM session is activated such that a CGM will continually measure glucose levels of the user for use in therapy calculations in the system. At step 304, it is determined if the pump is communicatively coupled with a remote device such as a smart phone, dedicated remote control, smart watch, etc. capable of receiving the CGM data in lieu of the pump. If so, then communications from the CGM are automatically conducted by that device. If it is determined the pump is not communicatively coupled with a remote device at step 306, then a wireless connection is established between the pump and the CGM at step 308 and subsequent communications from the CGM are sent to the pump at step 310. As indicated in the Figure, the system can regularly or continuously determine which connections are present and, in the event CGM communications are being sent to the pump, automatically cease such communications and connect the phone or other device to the CGM when a connection between the pump and that device is established. Similarly, if the pump becomes no longer connected with the remote device communicating with the CGM, and if no alternative remote devices are present for such communications, the pump can transition to communicating directly with the CGM.

In some embodiments, CGM communications can also be routed based on a power level of one or more devices in the system. For example, as noted above, the system can default to receiving CGM communications at a phone, smartwatch, or other device any time that the pump is connected to such a device. However, a phone, for example, can have access not only its own power level, but the power level of any other device with which it is communicatively coupled, including the pump, a smart watch etc. In the event that the phone (or other primary device) has a battery below a certain threshold, the phone can instead cause another device to connect to the CGM. For example, there may be a hierarchy of devices ranked according to communications preference, typically with the phone being the preferred device and the pump being the final device, such as, for example, phone, watch, pump. If the battery on the phone is below a certain threshold, then communications can be directed to the watch. If the batteries on the phone and the watch are below the threshold, communications can instead go directly to the pump. Such thresholds may be relative. For example, if the pump has a fully charged battery communications may be routed to the pump in lieu of the phone and/or other devices at a higher battery level of the phone, smartwatch, etc. than if the pump had a lower battery level.

Although routing of CGM communications to a device other than a pump is primarily described herein with respect to power savings for the pump battery, such methods can be employed for other purposes. For example, because there are multiple CGM manufacturers, such devices may use different communication modalities, such as, for example, different versions of Bluetooth, near field communication (NFC) etc. In some circumstances, a pump may not possess the necessary software or hardware to communicate with a CGM employing a particular technology. Typically, a phone will have greater communication capabilities and a greater ability to update software or other features to provide such capabilities. In such circumstances, if it is determined that a pump is not able to communicate with a CGM (or other device) due to a technical incompatibility or lack of capability of the pump, the communications can instead be sent to a phone, watch or other device in the system that is able to communicate with the pump and relay the information to the pump using a different communication protocol over which the pump is able to communicate.

CGM communications can also be routed based on a connectivity of various devices in the system. For example, commonly owned U.S. Patent Publication No. 2020/0206420, which is hereby incorporated by reference herein, discloses use of a phone, watch or other device to relay communications from a CGM to a pump when connectivity between the CGM and pump is lost due to, for example, the body interfering with wireless communications between the devices based on where the devices are positioned on the body. However, connectivity among other devices in the system can also warrant a modification to communication protocols. For example, a relative location of a CGM on the body and phone may cause difficulty in communication between the devices. In such a circumstance, the system can automatically route communications through another device, such as a watch, or can cause the pump to directly communicate with the CGM. In such embodiments, the system can automatically monitor signal strength for communications between the various devices in the system and transition from sending communications to one device to another device when connectivity between the CGM and a device falls below a threshold level and/or if connectivity with an alternative device is a threshold amount better than connectivity with a current device.

It should be noted that although embodiments herein are primarily described with routing of communications from a CGM, that the communication methodologies disclosed herein can relate to other devices in infusion pump systems that can have communications redirected from a pump to another device in the system. For example, the disclosure can relate to additional devices such as, for example, another analyte sensing device, an activity or other health monitor, heart rate monitor, etc.

In embodiments, a method of coordinating wireless communications in an infusion pump system including a continuous glucose monitoring (CGM) device, an infusion pump and a remote electronic device is described herein. A communication session can be initiated with the CGM device in which the CGM device measures glucose levels of a user for calculation of therapy parameters for delivery of insulin to the user with the infusion pump. It can then be determined if the infusion pump is communicatively coupled with the remote electronic device. Glucose levels can be transmitted from the CGM device to the remote electronic device if the infusion pump is communicatively coupled with the remote electronic device.

In an embodiment, glucose levels can be transmitted from the CGM device to the infusion pump if the infusion pump is not communicatively coupled with the remote electronic device.

In an embodiment, no communications occur between the infusion pump and the CGM device if the infusion pump is communicatively coupled with the remote electronic device.

In an embodiment, no communications occurring between the infusion pump and the CGM device if the infusion pump is communicatively coupled with the remote electronic device includes causing the infusion pump to not broadcast an advertisement data packet for detection by the CGM device.

In an embodiment, no communications occurring between the infusion pump and the CGM device if the infusion pump is communicatively coupled with the remote electronic device includes terminating any existing communicative coupling between the infusion pump and the CGM device.

In an embodiment, it can be detected that the infusion pump is no longer communicatively coupled with the remote electronic device and the infusion pump can then seek to establish connectivity with the CGM device.

In an embodiment, the remote electronic device can transmit a control command to the infusion pump for delivery of therapy with the infusion pump based on the glucose levels received by the remote electronic device from the CGM device.

In an embodiment, it can be determined that a battery level of the remote electronic device is below a threshold level and the CGM device can transmit the glucose levels to another device in response to the battery level of the remote electronic device being below the threshold level.

In an embodiment, transmitting the glucose levels to another device includes transmitting the glucose levels to the infusion pump.

In an embodiment, transmitting the glucose levels to another device includes transmitting the glucose levels to a second remote electronic device.

In embodiments, a method of coordinating wireless communications in an infusion pump system including a continuous glucose monitoring (CGM) device, an infusion pump and a remote electronic device is disclosed herein. A communication session can be initiated with the CGM device in which the CGM device measures glucose levels of a user for calculation of therapy parameters for delivery of insulin to the user with the infusion pump. If it is determined that the infusion pump is communicatively coupled with the remote electronic device, no communications occur between the infusion pump and the CGM device.

In embodiments, no communications occurring between the infusion pump and the CGM device if the infusion pump is communicatively coupled with the remote electronic device includes causing the infusion pump to not broadcast an advertisement data packet for detection by the CGM device.

In embodiments, no communications occurring between the infusion pump and the CGM device if the infusion pump is communicatively coupled with the remote electronic device includes terminating any existing communicative coupling between the infusion pump and the CGM device.

In an embodiment, glucose levels are received from the CGM device at the remote electronic device if the infusion pump is communicatively coupled with the remote electronic device.

In embodiments, the glucose levels are received from the CGM device at the infusion pump if the infusion pump is not communicatively coupled with the remote electronic device

In embodiments, the remote electronic device can be caused to transmit a control command to the infusion pump for delivery of therapy with the infusion pump based on the glucose levels received by the remote electronic device from the CGM device.

In an embodiment, if it is detected that the infusion pump is no longer communicatively coupled with the remote electronic device, the infusion pump can seek to establish connectivity with the CGM device.

In an embodiment, if it is determined that a battery level of the remote electronic device is below a threshold level the CGM device can establish communications with another device.

In an embodiment, the CGM device establishing communications with another device includes causing the CGM device to establish communications with the infusion pump.

In an embodiment, the CGM device establishing communications with another device includes causing the CGM device to establish communications with a second remote electronic device.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; 11,224,693; 11,291,763; 11,305,057; 11,458,246; 11,464,908; 11,654,236; and 11,911,595 and commonly owned U.S. Patent Publication Nos. 2009/0287180; 2012/0123230; 2013/0053816; 2014/0276423; 2014/0276569; 2014/0276570; 2018/0071454; 2019/0307952; 2020/0206420; 2020/0329433; 2020/0368430; 2020/0372995; 2021/0001044; 2021/0113766; 2022/0062553; 2022/0139522; 2022/0223250; 2022/0233772; 2022/0233773; 2022/0238201; 2022/0265927; 2023/0034408; 2022/0344017; 2022/0370708; 2022/0037465; 2023/0040677; 2023/0047034; 2023/0113545; 2023/0113755; 2023/0166037; 2023/0173170; 2023/0201452; 2023/0277765; 2023/0338653; 2023/0381406; 2024/0226423; 2024/0226424 and 2024/0277924 and commonly owned U.S. patent application Ser. Nos. 17/368,968; 17/896,492; 18/011,060; 18/071,835; 18/207,094; 18/398,543; 18/441,735; 18/474,839; 18/475,916; 18/478,552 and 18/678,130.

For purposes of interpreting the claims, it is expressly intended that the provisions of 35 U.S.C. § 112(f) are not to be invoked unless the specific terms “means for” or “step for” are recited in a claim.

1. A method of coordinating wireless communications in an infusion pump system including a continuous glucose monitoring (CGM) device, an infusion pump and a remote electronic device, comprising:
initiating a communication session with the CGM device in which the CGM device measures glucose levels of a user for calculation of therapy parameters for delivery of insulin to the user with the infusion pump;
determining if the infusion pump is communicatively coupled with the remote electronic device; and
receiving the glucose levels from the CGM device at the remote electronic device if the infusion pump is communicatively coupled with the remote electronic device.

2. The method of claim 1, further comprising receiving the glucose levels from the CGM device at the infusion pump if the infusion pump is not communicatively coupled with the remote electronic device.

3. The method of claim 1, further comprising causing no communications to occur between the infusion pump and the CGM device if the infusion pump is communicatively coupled with the remote electronic device.

4. The method of claim 3, wherein causing no communications to occur between the infusion pump and the CGM device if the infusion pump is communicatively coupled with the remote electronic device includes causing the infusion pump to not broadcast an advertisement data packet for detection by the CGM device.

5. The method of claim 3, wherein causing no communications to occur between the infusion pump and the CGM device if the infusion pump is communicatively coupled with the remote electronic device includes terminating any existing communicative coupling between the infusion pump and the CGM device.

6. The method of claim 1, further comprising:
detecting that the infusion pump is no longer communicatively coupled with the remote electronic device; and
causing the infusion pump to seek to establish connectivity with the CGM device when the infusion pump is no longer communicatively coupled with the remote electronic device.

7. The method of claim 1, further comprising causing the remote electronic device to transmit a control command to the infusion pump for delivery of therapy with the infusion pump based on the glucose levels received by the remote electronic device from the CGM device.

8. The method of claim 1, further comprising:
determining that a battery level of the remote electronic device is below a threshold level; and
causing the CGM device to transmit the glucose levels to another device in response to the battery level of the remote electronic device being below the threshold level.

9. The method of claim 8, wherein transmitting the glucose levels to another device includes transmitting the glucose levels to the infusion pump.

10. The method of claim 8, wherein transmitting the glucose levels to another device includes transmitting the glucose levels to a second remote electronic device.

11. A method of coordinating wireless communications in an infusion pump system including a continuous glucose monitoring (CGM) device, an infusion pump and a remote electronic device, comprising:
initiating a communication session with the CGM device in which the CGM device measures glucose levels of a user for calculation of therapy parameters for delivery of insulin to the user with the infusion pump;
determining if the infusion pump is communicatively coupled with the remote electronic device; and
causing no communications to occur between the infusion pump and the CGM device if the infusion pump is communicatively coupled with the remote electronic device.

12. The method of claim 11, wherein causing no communications to occur between the infusion pump and the CGM device if the infusion pump is communicatively coupled with the remote electronic device includes causing the infusion pump to not broadcast an advertisement data packet for detection by the CGM device.

13. The method of claim 11, wherein causing no communications to occur between the infusion pump and the CGM device if the infusion pump is communicatively coupled with the remote electronic device includes terminating any existing communicative coupling between the infusion pump and the CGM device.

14. The method of claim 11, further comprising receiving the glucose levels from the CGM device at the remote electronic device if the infusion pump is communicatively coupled with the remote electronic device.

15. The method of claim 14, further comprising receiving the glucose levels from the CGM device at the infusion pump if the infusion pump is not communicatively coupled with the remote electronic device.

16. The method of claim 14, further comprising causing the remote electronic device to transmit a control command to the infusion pump for delivery of therapy with the infusion pump based on the glucose levels received by the remote electronic device from the CGM device.

17. The method of claim 11, further comprising.
detecting that the infusion pump is no longer communicatively coupled with the remote electronic device; and
causing the infusion pump to seek to establish connectivity with the CGM device when the infusion pump is no longer communicatively coupled with the remote electronic device.

18. The method of claim 11, further comprising:
determining that a battery level of the remote electronic device is below a threshold level; and
causing the CGM device to establish communications with another device in response to the battery level of the remote electronic device being below the threshold level.

19. The method of claim 18, wherein causing the CGM device to establish communications with another device includes causing the CGM device to establish communications with the infusion pump.

20. The method of claim 18, wherein causing the CGM device to establish communications with another device includes causing the CGM device to establish communications with a second remote electronic device.
